(19) United States
(12) Patent Application Publication
(10) Pub. No.: US 2009/0304780 A1
(43) Pub. Date: Dec. 10, 2009

(54) WOUND DRESSINGS INCORPORATING HONEY

(76) Inventors: Albertus Justus Johannes van den Berg, Utrecht (NL); Matthias Johannes Hoekstra, Amsterdam (NL)

Correspondence Address: SPENCER FANE BRITT & BROWNE LLP, 1 NORTH BRENTWOOD BLVD., SUITE 1000, ST. LOUIS, MO, 63105-3925, US

(21) Appl. No.: 12/393,520

(22) Filed: Feb. 26, 2009

Related U.S. Application Data
(60) Provisional application No. 61/040,402, filed on Mar. 28, 2008.

(51) Int. Cl. A61K 9/00; A61K 35/00; A61P 17/02

(52) U.S. Cl. 424/447; 424/537

(57) ABSTRACT

An improved wound dressing using a honey composition is provided. Preferably, the honey composition includes buckwheat honey.

### RELATED APPLICATION

This application claims the benefit of U.S. Provisional Application 61/040,402, filed Mar. 28, 2008, entitled “Wound Dressings Incorporating Honey.”

### FIELD OF THE INVENTION

The present invention relates general to the use of honey for treatment of

wounds, and more specifically to wound dressings incorporating honey.

### BACKGROUND OF THE INVENTION

Buckwheat honey: nature's gift to wound treatment?

Honey, queen among sweeteners, is a complex mixture with glucose and fructose present as major components obtained from nectaring flowers, and enzymes added by the honey bee. Dependent of the floral source, honey contains various other minor constituents (phytochemicals). Unifloral honeys obtained from a single flowering plant species show specific characteristics which may determine their value for medicinal application. For instance, the potency of antibacterial factors has been reported to vary as much as 100-fold between honeys of different floral sources.^{1,2} In contrast to multifioral honey, different batches of a unifloral honey contain identical constituents in more or less similar concentrations, and show biological activities, which are of the same range. By better reliability of clinical efficacy as a consequence of greater consistency in composition, unifloral honeys are to be preferred for medicinal use.

Records of wounds being covered with honey date back to ancient Egypt, and Dioscorides (50 AD) reported honey to be ‘good for all rotten and hollow ulcers’.³ Honey has recently been ‘rediscovered’ by the medical profession^{1,4,5}, and many publications on clinical wound healing have stated that honey rapidly eradicates infection with no adverse effects.⁶⁻⁹ Moreover, it may significantly promote the wound healing process as observed in the clinic.¹ Research to support these clinical observations has mainly focused on honey's antibacterial properties, and has shown that certain types, such as manuka honey (obtained from Leptospermum scoparium), stop bacteria from growing—even strains resistant to methicillin (MRSA) and vancomycin (VRE).¹⁰

Reduced inflammation observed in the clinic following the application of honey is supported by histological evidence of reduced numbers of inflammatory cells present in the wound tissue.¹ Furthermore, pasture honey (multifloral) and unifloral manuka honey at concentrations of 1 % have been reported to stimulate monocytes in vitro to release tumor necrosis factor-alpha (TNF-α), a regulatory cytokine, which induces fibroblast collagen synthesis, thereby initiating healing.^{11, 12}

The non-adaptive division of the immune system, also known as the innate immune system, is immediately activated in response to foreign substances and invading microorganisms encountered, thus playing a role in the wound-healing process, as well.¹³ The cellular part of this first line of defense is constituted by polymorphonuclear neutrophils (PMNs), macrophages and monocytes. In activated PMNs, a cell-membrane located multi-component NADPH oxidase is assembled from separate cytosolic subunits, which generates superoxide anion (O₂^{•-}) by transfer of electrons to oxygen.¹⁴ Superoxide anion produced by activated PMNs recruited to the wound site is converted into hydrogen peroxide (H₂O₂).¹⁴⁻¹⁶ Myeloperoxidase (MPO), an enzyme released by activated PMNs, catalyses the conversion of hydrogen peroxide into hypochlorite anion (OCl⁻), a potent bactericidal agent that kills invading microorganisms.¹⁷ When ferrous ion (Fe²⁺) is present, hydrogen peroxide may also be converted into hydroxyl radical (OH^{•}), a very strong but short-lived oxidant.¹⁸ The free radicals (superoxide anion and hydroxyl radical) and the non-radicals (hydrogen peroxide and hypochlorite anion) are collectively known as reactive oxygen species (ROS).

Activation of PMNs results in both intracellular and extracellular generation of ROS. Intracellularly, the combined action of ROS produced within the phagolysosome and proteolytic enzymes released from the granules (lysosomes) kills ingested bacteria, preventing wound infection.^{15,16} Extracellularly, however, excessive generation of ROS has detrimental effects on surrounding tissue. Superoxide anion easily reacts with nitric oxide (NO^{•}), a radical produced, for instance, by macrophages at the wound site. This results in the formation of peroxynitrite (ONOO⁻) which, like hydroxyl radical and hypochlorite anion, is another major oxidant causing tissue damage.¹⁹

Reactive oxygen species play an important role in impaired wound

healing.²⁰⁻²² Although in some cases ROS are considered to possess certain beneficial antimicrobial properties and second messenger abilities²³, prolonged exposure to elevated levels of ROS causes cell damage and may eventually inhibit healing of both acute and chronic wounds. Typically, burn injuries show excessive activity of free radicals.^{24,25} Jambolan honey (from Syzygium cumini), selected on basis of high antibacterial activity has been suggested to initiate healing by free radical control as part of honey's antioxidant activity.²⁴ Free radicals have been implicated in hypertrophic scar formation following burn injuries.²⁴ Therefore, it is likely that reducing levels of free radicals and other oxidants in the wound bed will aid wound management. So, we assessed the antioxidant and anti-inflammatory properties of several honeys of different floral sources using in vitro assays for testing their ability to inhibit ROS production by activated human PMNs and to scavenge superoxide anion in a cell-free system.

Complement is part of the humoral response of the innate immune system to foreign matters.²⁶ Complement activation proceeds through a series of proteolytic steps in either the classical, alternative or lectin pathway. The terminal stage of each pathway results in the formation of a macromolecular membrane attack complex (MAC) that kills invading microorganisms through lysis.²⁷ Complement factor C3b is an opsonin, a molecule acting as an enhancer of phagocytosis by binding to foreign cells. Since PMNs have receptors for C3b, these phagocytic cells can easily detect, ingest and destroy opsonized microorganisms.²⁸ In addition, the complement cascade results in the generation of small split products mediating many immunoregulatory effects, such as complement factor C5a. The latter activates PMNs, and also attracts these cells to the wound site.²⁹ Inhibition of complement may therefore result in lower wound levels of ROS by limiting the factors involved in PMN recruitment and activation. Therefore, in addition to the antioxidant activities mentioned above, we tested different varieties of honey also for their ability to inhibit activated human complement.

There is a long-felt, but unmet, need for improved wound dressings. Accordingly, it would be advantageous to provide for wound dressings that inhibit ROS activity.

### SUMMARY OF THE INVENTION

Hydroxyl radical and hypochlorite anion formed at the wound site from superoxide anion produced by activated polymorphonuclear neutrophils (PMNs) are considered important factors in impaired wound healing. Superoxide anion may also react with nitric oxide produced by macrophages to form peroxynitrite, a third strong oxidant that damages surrounding tissue. For selection of honey to be used in wound-healing products, different samples were compared for their capacity to reduce levels of reactive oxygen species (ROS), in vitro.

Honey samples were tested in assays for inhibition of ROS production by activated human PMNs, antioxidant activity (scavenging of superoxide anion in a cell-free system), and inhibition of human complement (reducing levels of ROS by limiting formation of complement factors that attract and stimulate PMNs). For buckwheat honey (USA, NY), contents of moisture and free acids were determined by refractive index measurement, and potentiometric titration, respectively. Honey constituents other than sugars were investigated by TLC (thin layer chromatography) using natural product reagent for detection of phenolic compounds; constituents with antioxidant properties were detected by spraying the chromatogram with DPPH.

Although most honey samples were shown to be active, significant differences were observed, activities of highly active honey exceeding activities of samples with minor effects by factor 4 to 30. Most pronounced activities were found for American buckwheat honey from the state of New York. Phenolic constituents of buckwheat honey were shown to have antioxidant activity.

Buckwheat honey being superior in reducing levels of reactive oxygen

species (ROS) was selected for use in wound-healing products. Major antioxidant principles in buckwheat honey are phenolic constituents present in relatively large amounts. Phenolic compounds may also exert antibacterial activity, whereas low pH and high free acid content as determined for buckwheat honey selected may be other factors beneficial to healing of wounds.

Declaration of interest: Research was financed by Greystone Medical Group.

### DETAILED DESCRIPTION

Honeys of different floral sources were tested for their ability to reduce oxidant levels as described above, in order to select the most active variety in this respect for use in wound-management products to be developed. Testing included two varieties, which had already been applied in commercial wound-healing products, i.e. 1) unifloral manuka honey, highly valued for its medicinal properties and subject of several studies by the scientific community^{1,7,10}, and 2) special multifloral honey from Chile, selected by Sociedad Apicola Verkruisen for application to wounds on basis of high glucose oxidase content (as will be explained in the discussion section). Other varieties tested concerned two samples of American buckwheat honey very dark-brown in color, and two exotic varieties coming from Hawaii, i.e. brown macadamia honey, and almost white rare kiawe honey. Finally, a Canadian mixture of clover and alfalfa honey as another representative of light-colored types besides Hawaiian kiawe honey completed the series.

Materials and Methods

Honey Samples Tested

A mixture of clover (Trifolium species) and alfalfa (Medicago saliva)

honey came from Canada (Golden Acres Co,, Three Hills, Alberta), and was coded Ca. Two samples of American buckwheat honey (Fagopyrum esculentum), AA and AB, supplied by bee keepers in the states of New York and North Dakota, respectively, were obtained from Dutch Gold Honey Inc. (Lancaster, Pa., U.S.A.). Chilean honey (Ch) from the Maule Region, Miel de Abeja organica, was received from Sociedad Apicola Verkruisen (San Javier, Chile). Active manuka (Leptospermum scoparium) honey (M), supplied by Comvita (Bay of Plenty) was purchased in New Zealand. From Hawaii two samples were obtained, i.e. HA, macadamia honey (Macadamia integrifolia; Hawaii Island Honey Co.), and HB, kiawe honey (Prosopis pallida) supplied by Volcano Island Co.

Effect of Honey—Inhibition of Factors Increasing Oxidant Levels

Different varieties of honey were tested in bioassays, which are commonly used for anti-inflammatory activity screening. The following bioassays were performed, which have been described in detail, previously.³⁰

Honey Investigated as Inhibitor of ROS Produced by PMNs Using a Luminol-Dependent Chemiluminescence Assay

This chemiluminescence assay concerns physiologically relevant in vitro assessment of potential anti-inflammatory effects on basis of inhibition of ROS production by inflammatory cells. Honey samples were tested for their ability to inhibit production of reactive oxygen species (ROS) by zymosan-activated human neutrophils (PMNs) using luminol as chemiluminescent probe. Reaction of luminol with ROS, in particular hypochlorite anion (OCl³¹), results in formation of an excited oxidation product, which emits light (chemiluminescence) upon returning to its ground state.

Briefly, in a 96-wells plate, freshly isolated human PMNs³¹ were stimulated by addition of opsonized zymosan. The resulting production of ROS was detected as luminol-dependent chemiluminescence using a Titertek Luminoskan luminometer. Chemiluminescence was monitored for 0.5 seconds every two minutes over a 30-minute period at 37° C. Peak levels of chemiluminesence measured for honey samples and controls (identical incubations without honey present) were used to calculate inhibition of ROS production.³²

Investigating Honey's Capacity to Scavenge Superoxide Anion Using Lucigenin-Dependent Chemiluminesence

Although not causing tissue damage as such, superoxide anion (O₂^{•-}) may eventually lead to detrimental effects by conversion of O₂^{•-} into hydroxyl radical, hypochlorite anion, and/or peroxynitrite. Thus, scavenging of superoxide anion may contribute to honey's wound-healing properties.

Different from the assay mentioned above, which is primarily focused on inhibition of ROS production, thus PMN functioning, the scavenging assay concerns a situation with superoxide anion already present. The capacity of honey samples to scavenge superoxide anion was determined in a cell-free system as inhibition of chemiluminesence; lucigenin was used as chemiluminescent marker, specifically detecting superoxide anion.

Briefly, in a 96-wells plate, production of superoxide anion was initiated by addition of xanthine oxidase to a mixture of hypoxanthine, lucigenin and honey sample. The chemilumineseence signal generated was monitored for 0.5 seconds every three minutes during 30 minutes, using a Fluoroskan Ascent FL luminometer. Ultimately, activity of the honey samples was calculated from the part of the chemilumineseence signal that was inhibited by superoxide dismutase (SOD).³⁰

Testing Honey for Inhibition of Human Complement Using a Hemolytic Assay

Activity of different honey varieties concerned inhibition of human complement activated via the classical pathway, and was determined using a microtiter plate-method.³³ Briefly, serial dilutions of honey samples were prepared in veronal buffer supplemented with calcium (Ca²⁺) and magnesium ions (Mg²⁺), and incubated with human pooled serum (HPS) as source of complement for 30 minutes. Subsequently, sheep erythrocytes (ShE) were added, which had been sensitized by incubation with amboceptor, as classical pathway complement activity can only be triggered by particles or cells coated with immunoglobulins. Activation of complement eventually results in formation of MAC (membrane attach complex) causing lysis of ShE. The amount of hemoglobin released, spectrophotometrically determined at 405 nm using an automatic ELISA reader, served as measure for classical pathway complement activity.

Presentation of Activities Determined in Bioassays—RIC50

In general, inhibitory activity is expressed as IC50, which is the amount of sample per ml (mg/ml) in the test system (bioassay) giving 50% inhibition, thus low IC50 values representing strong activities. However, diagrams showing test results as IC50 often cause confusion by low bars indicating high activity and high bars representing minor effects. Therefore, activities have been presented here as RIC50 (Reciprocal IC50), 1/IC50 in ml/g, which is the volume (ml) to be added per gram of sample giving 50% inhibition. In this way, samples with stronger activities requiring more dilution (increased volume) show higher RIC50values. High bars now corresponding with strong activities greatly improves interpretation of test results as presented in bar diagrams. RIC50 values presented are the mean±standard deviation (SD).

Physical Characteristics of Buckwheat Honey

Determination of Moisture

To prevent growth of high osmolarity-resistant yeast species, the water content of honey should not be more than 20%. Although eventual honey products may be gamma-ray sterilized, growth of yeasts prior to sterilization may affect honey's medicinal properties. The amount of water present was determined according to the European Pharmacopoeia³⁴ by measurement of refractive index (RI), which increases with increasing sugar concentration. Moisture content was derived from RI corrected for temperature (+0.00023/° C.), using a table of RI values (at 20.0° C.) with corresponding water content listed.

Potentiometric Measurement of pH

Since acidity may be a factor contributing to wound healing, pH and free

acid content were also determined. A solution of 25 gram of buckwheat honey in 75 ml of carbon dioxide-free demineralized water was used for determination of pH. The latter was measured as the potential difference (voltage) between a glass electrode and silver chloride (AgCl) reference electrode as described in the European Pharmacopoeia.³⁴

Free Acid Content

Free acids were quantified by potentiometric acid-base titration with potassium hydroxide solution of known concentration as described in Harmonized Methods of the International Honey Commission.³⁵ In general, the end-point is determined using a visual indicator such as phenolphthalein, colorless in acid solution, but turning into pink above pH 8.2. However, for buckwheat honey with its dark-brown color, a visual indicator could not be used. Therefore, the point of equivalence was determined at pH 8.3 by potentiometric measurement (see above). Free acid content of honey is expressed in meq/kg (milliequivalents/kg), which is the volume (in ml) of 1 M potassium hydroxide solution to be added to 1000 g of honey for neutralization of acids present.

Investigation of Phenolic Constituents by Thin Layer Chromatography (TLC)

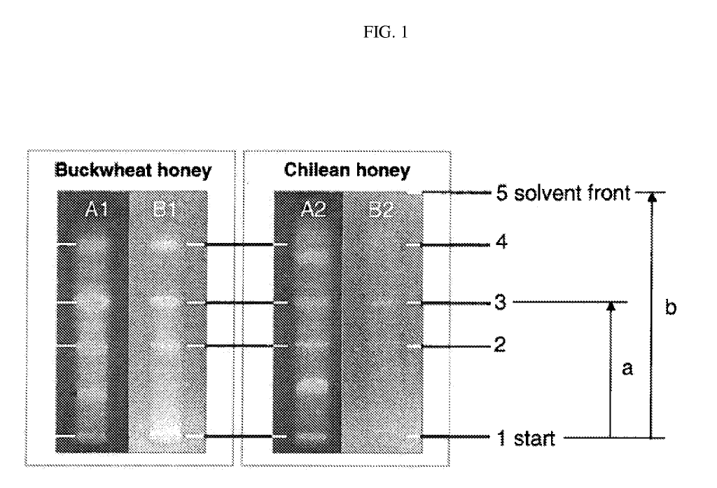

Thin layer chromatography is a rapid analytical method for detection of separate compounds present in complex mixtures. After application of a multicomponent sample to a TLC plate at the start position, components of interest are separated by a solvent (mobile phase) migrating through the thin sorbent layer (stationary phase). The retention factor or Rᵣ is defined as the traveling distance by a compound from the start (FIG. 1: a) divided by the distance allowed for solvent migration (from start to solvent front; FIG. 1: b). Constituents are detected as colored spots under ultraviolet light and/or after spraying the chromatogram with specific reagents (FIG. 1). Thus, TLC enables characterization of compounds by R_{f} value and specific color reactions.

Extraction of Phenolic Components From Honey

Consisting of mainly sugars, honey as such cannot be subjected to TLC

analysis of phenolic components, directly. The latter constituents present in minor amounts can only be detected after concentration and without sugars interfering. Therefore, phenolic constituents were extracted from honey prior to TLC analysis, as follows: Honey (25 g) was dissolved in 75 ml of water, and the solution was extracted with ethyl acetate (3×50ml). The combined ethyl acetate extracts were dried over anhydrous magnesium sulfate. After filtration, and evaporation of the solvent under reduced pressure, the residue was taken up in 10 ml of ethanol (96%) and subjected to TLC analysis.

TLC Analysis of Phenolic Constituents

Ethanol solutions containing phenolic constituents (see above) extracted from American NY buckwheat honey (AA) and Chilean honey (Ch) were applied in 30 μl amounts to TLC plates pre-coated with silica gel. The solvent system used was acetonitrile/water (87:13; saturated chamber). After development of the chromatogram, the plate was sprayed with NPR (natural product reagent; 2-aminoethyldiphenyl borinate), and examined under ultraviolet light of 366 nm. NPR specifically detects phenolic constituents, including flavonoids, which become visible as blue and green fluorescent spots. Spraying (another) chromatogram with DPPH (2,2-diphenyl-1-picrylhydrazyl radical; 0.2 mg/ml dissolved in absolute ethanol) showed the presence of constituents with antioxidant activity as yellow spots on a purple background (examination in daylight). DPPH is a relatively stable purple-colored radical that turns into yellow DPPHH by acceptance of hydrogen atom donated by antioxidants.

Results

Biological Activities

Different honey varieties were tested in vitro for activities leading to

reduction of oxidant levels, i.e. inhibition of ROS production, scavenging of superoxide anion, and inhibition of human complement.

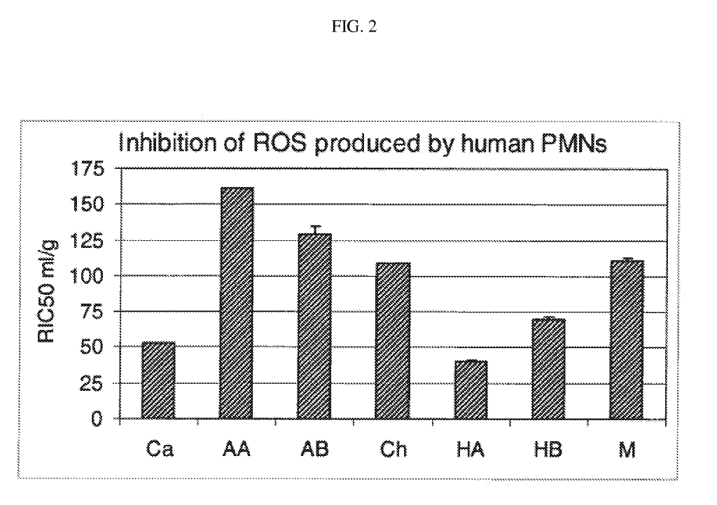

All honey samples tested showed inhibition in the bioassay for ROS produced by activated human PMNs with activities ranging from 160 and 130 ml/g (buckwheat honey from New York, and North Dakota, respectively) to 40 ml/g (Hawaiian kiawe honey) as shown in FIG. 2. No differences were found between Chilean and manuka honey showing a RIC50 value around 110 ml/g.

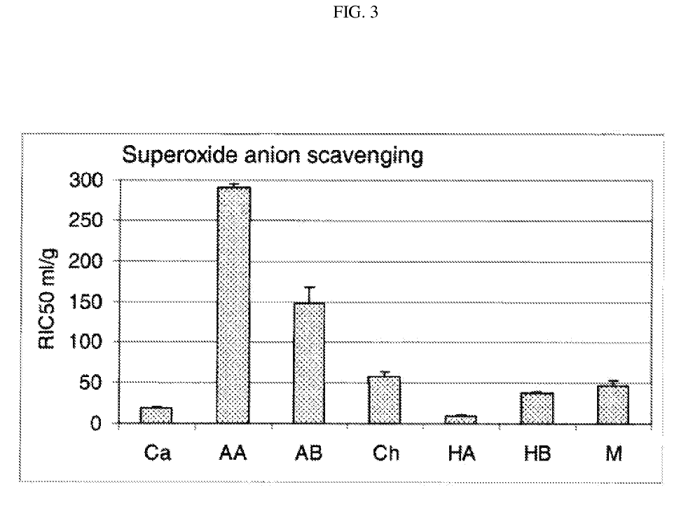

Differences in superoxide anion scavenging capacity were found to be much more pronounced (FIG. 3). Again, highest activity by far was shown by NY buckwheat honey (RIC50: 290 ml/g), exceeding the RIC50 value of least active Hawaiian kiawe honey (10 ml/g) by factor almost 30. Although showing half the activity as determined for the New York sample, in comparison with the other samples investigated superoxide anion scavenging capacity of North Dakota buckwheat honey (RIC50: 150 ml/g) can still be considered strong. No significant difference was found between activities determined for Chilean (59 ml/g) and manuka honey (48 ml/g).

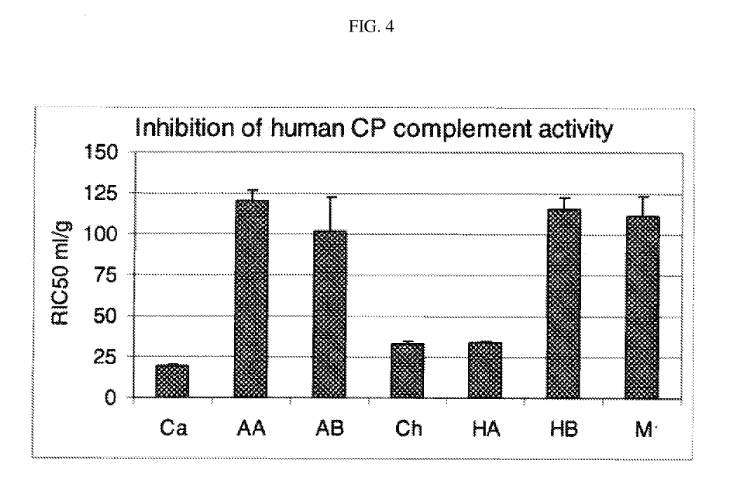

Results of testing honey samples for inhibition of human classical pathway (CP) complement activity are presented in FIG. 4. Although the strongest inhibition was shown by NY buckwheat honey (RIC50: 120 ml/g), this activity was not significantly different from ND buckwheat (102 ml/g), Hawaiian macadamia (115 ml/g) and manuka honey (111 ml/g). In this assay, the Chilean sample (RIC: 33 ml/g) was found to score worse than manuka, and equal to Hawaiian kiawe honey (RIC50: 34 ml/ml).

American buckwheat honey from New York showed the most pronounced activities (FIG. 2-4), that is the inhibition of human complement, which results in reduced formation of factors that attract and stimulate PMNs, as well as scavenging of oxidants produced by PMNs upon activation. The in vitro results obtained were encouraging and honey may exert these activities in vivo, as well. However, this was not assessed in this study.

On basis of superior in vitro activities, and after it had been established that heavy metals, pesticides and antibiotics were all below detection limits or present in acceptable amounts, NY buckwheat honey was selected to be used in wound-healing products, i.e. MelMax® and MelDra® (Dermagenics Europe BV, Kaatsheuvel, The Netherlands), and subjected to further research.

Physical Characteristics of NY Buckwheat Honey

In addition to testing for biological activities resulting in decreased levels of radicals and oxidants, and analysis of contaminants for safety purposes, some other parameters were determined that may also contribute to wound-healing effects of NY buckwheat honey, and quality of eventual products.

Moisture Content

Refractive index (RI) of NY buckwheat honey measured at 22.1° C. was N_{D} 1.4939. Correction for temperature resulted in N_{D} 1.4944, corresponding with a moisture content of 16.8% (w/w), which is perfectly below the upper limit of 20% as required by European legislation³⁶, and European Pharmacopoeia³⁴.

Acidity

NY buckwheat honey was found to have relatively strong acid properties, represented by low pH and high free acid content. Measurement of pH using a solution of NY buckwheat honey in water (25% w/v) showed pH 3.3, which is considered low in comparison with the average pH 3.9 for non-tropical honeys with a typical range of 3.4 to 6.1.³⁷

The pH represents-¹⁰log [H⁺], [H⁺] being the concentration of protons (H⁺ions) in moles per liter. Free acid content is the amount of 1 M potassium hydroxide solution (in ml) required for neutralization of acid components present in 1 kg of honey. In contrast to strong mineral acids (e.g. sulfuric acid), acids in honey concern weak organic acids only capable of limited dissociation into protons, which results in lower |H⁺|thus higher pH values. Neutralization by potassium hydroxide results in complete dissociation, however. So, free acid content concerns components being capable to split off protons, whereas pH is a measure for protons being actually present. In other words, the free acid content of honey is higher than can be derived from its pH measured.

Determination of free acids in NY buckwheat honey showed a content of 50 meq/kg, which is exactly the maximum allowed by European legislation towards honey used tor consumption.³⁶

TLC Analysis

Thin layer chromatography analysis with natural product reagent detection

showed blue and green-fluorescent spots, indicating phenolic compounds, including flavonoids, extracted from NY buckwheat honey (FIG. 1: TLC-A1). Detection of antioxidants by DPPH showed the presence of some strong antioxidant constituents that immediately turned purple DPPH into yellow (FIG. 1: TLC-B1).

In FIG. 1, A1 and A2, and B1 and B2 refer to pans of two separate

chromatograms A and B, which were developed under similar experimental conditions. By this, identical constituents of NY buckwheat honey on TLC-A1 and TLC-B1, will have the same Rf value as indicated by 1-4 (FIG. 1). Since fluorescent constituents were found in this respect to correspond with those detected by DPPH, phenolic compounds present in NY buckwheat honey were concluded to be major antioxidant principles, and such may account as well for antibacterial activity.³⁸

In addition, it was found that amounts of antioxidants in NY buckwheat honey (FIG. 1: TLC-B1) greatly outmeasured those present in Chilean honey (FIG. 1: TLC-B2). The component indicated by 3 with R_{f} 0.53 was found to be major antioxidant constituent of both NY buckwheat and Chilean honey, although the latter contained a relatively minor amount, only slightly reducing DPPH as observed by TLC (FIG. 1: TLC-B2). The Chilean honey investigated has also been used in wound care products, e.g. HoneySoft (Mediprof B V, Rijswijk, The Netherlands), but was selected on basis of high glucose oxidase content.

Discussion

NY Buckwheat Honey Selected on Basis of Pronounced in vitro Activities

Since elevated levels of reactive oxygen species may cause cell damage and inhibition of wound healing^{24,25}, honeys of different floral sources were tested in vitro for activities, which lower levels of oxidants, radicals included. On this basis NY buckwheat honey showing superior effects was selected for medicinal application. In comparison with manuka and Chilean honey, which have both been used in commercial wound-healing products, NY buckwheat honey was found to be a most strong scavenger of superoxide anion, in particular (FIG. 3), and most effective inhibitor of ROS produced by stimulated human PMNs (FIG. 2), NY buckwheat honey was also shown to inhibit classical pathway complement activity (FIG. 4), which results in limited formation of complement factors attracting and activating PMNs. In the latter assay, however, pronounced inhibition was not restricted to NY buckwheat honey, only, as ND buckwheat. Hawaiian macadamia, and manuka honey showed similar activities with differences not being significant.

Quality Control of NY Buckwheat Honey

Strict requirements regarding heavy metals, pesticides and antibiotics present have neither been included in the honey monograph of the European Pharmacopoeia³⁴, nor formulated for honey as food by European legislation³⁶. Nevertheless, to guarantee safety for wound-healing products eventually produced, NY buckwheat honey was tested for these contaminants, which were below detection limits or present in the amounts allowed for other food products.

Concerning quality, another parameter determined was moisture content. With 16.8% (w/w) water present, NY buckwheat honey amply met the legislatory requirements. As consequence of its high osmotic value (high sugar content) in general, honey draws wound exudate to the wound surface, thus creating a moist environment, which results in a non-adherent interface between dressing and wound bed.⁷ Reports on the significance of high osmolarity for antibacterial activity of honey are controversial (see below). However, moisture content was not determined as antibacterial factor of NY buckwheat honey to be applied, but as control to exclude high-osmolarity resistant yeasts having affected its properties, prior to being processed into wound-healing products.

Antibacterial Activity and Other Factors Promoting Wound Healing

The value of honey as an antibacterial has since long been recognized. Hydrogen peroxide—already present (residual) and/or generated by glucose oxidase activity upon dilution of honey—as well as phenolic constituents are considered major antibacterial factors.^{1,38} Neither glucose oxidase, nor residual hydrogen peroxide could be detected in NY buckwheat honey selected for application to wounds. With sugar contents above 80% (w/w), the high osmolarity of undiluted honey is sufficient to stop all microbial growth.¹ In practice, however, this may be of minor importance. Dependent of the type of wound, dilution by exudate may eventually result in loss of osmotic antibacterial activity.^{1,7} Somewhat contradictory, high osmolarity has been claimed responsible for honey's antimicrobial activity, if accompanied by low pH.^{39,40} However, not pH, but free acid content of non-peroxide honey has been found to significantly correlate with antibacterial activity against Staphylococcus aureus and Micrococcus luteus.⁴¹

In comparison with the average pH 3.9 reported for non-tropical honeys

with pH ranging from 3.4 to 6.1³⁷, pH 3.3 as measured for NY buckwheat honey can be considered low. Furthermore, its free acid content was determined to be the maximum allowed by European legislation³⁶, being 50 meq/kg. Referring to above, the low value for pH, but even more the high free acid content⁴¹ may be characteristics of NY buckwheat honey, contributing to antibacterial activity.

Lysed concentrates of platelets, pre-incubated at close to pH 5.0 (as opposed to pH 7.0), have been found to contain increased levels of platelet-derived growth factor (PDGF), and showed increased capacity to stimulate fibroblast proliferation in vitro.⁴² Although such has not been studied, showing relatively strong acidic properties, NY buckwheat honey may promote healing of chronic wounds by effecting a low pH at the wound site, resulting in fibroblast proliferation.

Compared to other types of honey, buckwheat honey is a rich source of phenolic antioxidants.⁴³ Since phenolic compounds also have antibacterial activity,³⁸ lack of glucose oxidase activity or hydrogen peroxide in NY buckwheat honey may well be compensated by phenolic constituents being present in relatively large amounts.

Pilot Clinical Studies

After selection based on in vitro biological activity, and additional research regarding quality aspects, as described above, NY buckwheat honey was used in wound-healing products, which were tested in pilot clinical experiments.

In a study including 21 burn patients with difficult to treat wounds, application of MelMax® (a wound dressing impregnated with NY buckwheat honey and an ointment containing a synthetic blend of metal ions and citric acid³⁰) resulted in wound closure and control of microbial contamination.⁴⁴ In another study including 60 patients, it was shown that treatment of venous leg ulcers with MelMax® showed similar results as silver-based dressings, or even better.⁴⁵

Concluding Remarks

As compared to honeys from some other floral sources, NY buckwheat honey showed pronounced in vitro activities leading to decreased levels of oxidants, radicals included. Antioxidant principles in NY buckwheat honey are constituted by phenolic constituents, in particular. The relatively low pH and high free acid content determined for NY buckwheat honey are other characteristic features, which may also contribute to healing of chronic wounds. Initial clinical pilot experiments have shown that MelMax® containing NY buckwheat honey was successfully applied to burn wounds and venous leg ulcers. Although often mentioned as major wound-healing factors, residual hydrogen peroxide, or glucose oxidase could not be detected. Instead, buckwheat honey was found to contain large amounts of antioxidant phenolic constituents that may also have antibacterial activity, thus compensating absence of hydrogen peroxide or its generation by glucose oxidase.

In wound management, bacteria resistant to antibiotics are becoming an increasing problem. Although results obtained so far are most promising, and indicate NY buckwheat honey to be an effective antimicrobial wound-healing product, final proof will only be provided by full clinical trials.

### REFERENCES

1 Molan P C. Re-introducing Honey in the Management of Wounds and Ulcers—Theory and Practice. Ostomy/Wound Management 2002; 48: 28-40.
2 Molan P C. The antibacterial activity of honey. 2. Variation in the potency of the antibacterial activity. Bee World 1992; 73: 59-76.
3 Gunther R T. The Greek Herbal of Dioscorides. New York, NY: Hafner; 1934 (reprinted 1959).
4 Namias N. Honey in the Management of Infections, Surg Infect 2003; 4: 219-26.
5 English H K, Pack A R, Molan P C. The effects of manuka honey on plaque and gingivitis: a pilot study. J Int Acad Periodontol 2004; 6: 63-7.
6 Dunford C. The use of honey-derived dressings to promote effective wound management. Prof Nurse 2005; 20: 35-8.
7 Molan P C, Belts J A. Clinical usage of honey as a wound dressing: an update. J Wound Care 2004; 13: 353-6.
8 Fox C. Honey as a dressing for chronic wounds in adults. Br J Community Nurs 2002; 7:530-4.
9 Gunes Y, Eser I. Effectiveness of a honey dressing for healing pressure ulcers. J Wound Ostomy Continence Nurs 2007; 34: 184-90.
10 Cooper R A, Molan P C, Harding K G. The sensitivity to honey of Gram-positive cocci of clinical significance isolated from wounds. J Applied Microbiol 2002; 93: 857-63.
11 Tonks A, Cooper R A, Price A J, et al. Stimulation of TNF-α release in monocytes by honey. Cytokine 2001; 14: 240-2.
12 Schultz G S, Mast B A, Molecular analysis of the environment of healing and chronic wounds: cytokines, proteases, and growth factors. Wounds 1998; 10 Supplement: 1-9F.
13 Tsirogianni A K, Moutsopoulos N M, Moutsopoulos H M. Wound healing: immunological aspects. Injury 2006; 37 (suppl. 1): S5-12.
14 Babior B M, Curnutte J T, McMurrich B J. The particulate superoxide-forming system from human neutrophils. Properties of the system and further evidence supporting its participation in the respiratory burst. J Clin Invest 1976; 58: 989-96.
15 Babior B M, Oxygen-dependent microbial killing by phagocytes. N Eng J Med 1978; 298: 659-68.
16 Hampton M B, Kettle A J, Winterboum C C. Inside the neutrophil phagosome: oxidants, myeloperoxidase, and bacterial killing. Blood 1998; 92: 3007-17.
17 Harrison J E, Shultz J. Studies on the chlorinating activity of myeloperoxidase. J Biol Chem 1976; 251: 1371-74.
18 Samuni A, Black C D V, Krishna C M, et al. Hydroxyl radical production by stimulated neutrophils reappraised. J Biol Chem 1988; 263: 13797-801.
19 Fukuyama N, Ichimori K, Su Z, et al. Peroxynitrite formation from activated human neutrophils. Biochem Biophys Res Commun 1996; 224: 414-9.
20 Latha B, Babu M. The involvement of free radicals in burn injury: a review. Burns 2001; 27: 309-17.
21 Thang P T, Patrick S, Teik L S, et al. Anti-oxidant effects of the extracts from the leaves of Chromolaena odorata on human dermal fibroblasts and epidermal keratinocytes against hydrogen peroxide and hypoxanthine-xanthine oxidase induced damage. Burns 2001; 27: 319-27.
22 Galeano M, Torre V, Deodato B, et al. Raxofelast, a hydrophilic vitamin E-like antioxidant stimulates wound healing in genetically diabetic mice. Surgery 2001; 129: 467-77.
23 Gordillo G M, Sen C K. Revisiting the essential role of oxygen in wound healing. Am J Surg 2003; 186: 259-63.
24 Subrahmanyarn M, Shahapure A G, Naganer N S, et al. Free radical control—the main mechanism of the action of honey in burns. Ann Burns Fire Disasters 2003; 16: 135-7.
25 Wan K C, Evans J H. Free radical involvement in hypertrophic scar formation. Free Rad Biol Med 1999; 26: 603-8.
26 Law S K A, Reid K B M. Complement: In focus (22nd edn). Oxford University Press, 1995.
27 Müller-Eberhard H J. The membrane attack complex of complement. Ann Rev Immunol 1986; 4: 503-28.
28 Gordon D L, Hostetter M K. Complement and host defence against microorganisms. Pathology 1986; 18: 365-75.
29 Hugli T E, Morgan E L. Mechanisms of leukocyte regulation by complement-derived factors. In: Regulation of leukocyte function. Snyderman, R. (Ed), New York: Plenum Press, 1984.
30 Van den Berg A J J, Halkes S B A, Quarles van Ufford H C, et al. A novel formulation of metal ions and citric acid reduces reactive oxygen species in vitro. J Wound Care 2003; 12; 413-8.
31 Verbrugh H A, Peters R, Peterson P K, et al. Phagocytosis and killing of staphylococci by polymorphonuclear and mononuclear leukocytes. J Clin Pathol 1978; 31: 539-45.
32 Van den Worm E, Beukelman C J, Van den Berg A J J, et al. Effects of apocynin and analogs on the inhibition of reactive oxygen species production by stimulated human neutrophils. Eur J Pharmacol 2001; 433: 225-30.
33 Klerx J P A M, Beukelman C J, Van Dijk H, et al. Microassay for colorimetric estimation of complement activity in guinea pig, human and mouse serum. J Immunol Methods 1983; 63: 215-20.
34 Monograph Honey—Mel 04/2005:2051. European Pharmacopoeia, Supplement 5.1. Council of Europe, Strasbourg—France, 2004,
35 Harmonized Methods. International Honey Commission, 2002; www.apis.admin.ch
36 Warenwetbesluit Honing. Staatsblad van het Koninkrijk der Nederlanden 2003; 492: 1-11.
37 Reference Guide to Nature's Sweetener. National Honey Board 2005; www.honey.com
38 Weston R J, Mitchell K R, Allen K L. Antibacterial phenolic components of New Zealand manuka honey. Food Chem 3 999; 64: 295-301.
39 Yatsunami K, Echigo T. Antibacterial activity of honey and royal jelly. Honeybee Science 1984; 5: 125-30.
40 Snowdon J A, Cliver D O. Microorganisms in honey. Int J Food Microbiol 1996; 31: 1-26.
41 Bogdanov S. Nature and Origin of the Antibacterial Substances in Honey. Lebensm Wiss Technol 1997; 30: 748-53.
42 Liu Y, Kalén A, Risto O, Wahlström O. Fibroblast proliferation due to exposure to a platelet concentrate in vitro is pH dependent. Wound Repair and Regeneration 2002; 10: 336-40.
43 Schramm D D, Karim M, Schrader H R. Honey with high levels of antioxidants can provide protection to healthy human subjects. J Agric Food Chem 2003; 51: 1732-35.
44 Hoeksema H, Pirayesh A, Hoekstra M J, et al. A clinical prospective study assessing the efficacy of a phenolic-rich honey and PH1-5 dressing in therapy resistant and colonized burn defects. 11th European Burns Association Congress, Estoril-Portugal, September 2005.
45 Rucigaj T P. Comparative effects of polyhydrated ionogens/honey-based dressings and silver-based dressings on the healing of venous leg ulcers. 16th Annual meeting, European Tissue Repair Society. Innovation in tissue repair: from the lab to the patient. Pisa-Italy, September 2006.

What is claimed is:

1. A wound dressing comprising:
a dressing material; and
a buckwheat honey composition applied to the dressing material, wherein the buckwheat honey composition comprises
buckwheat honey; and
a carrier comprising metal ions and citric acid.

2. The wound dressing of claim 1, wherein the dressing material comprises a bandage.

3. The wound dressing of claim 2, wherein the bandage is an acetate bandage.

4. The wound dressing of claim 1, wherein the buckwheat honey comprises New York buckwheat honey.

5. The wound dressing of claim 1, wherein the buckwheat honey includes water content of not more than 20%.

6. The wound dressing of claim 1, wherein the buckwheat honey composition is approximately 4 grams.

7. A wound dressing comprising:
a dressing material; and
a honey composition applied to the dressing material, wherein the honey composition comprises
honey having a water content of not more than 20%; and
a carrier comprising metal ions and citric acid.

8. The wound dressing of claim 7, wherein the dressing material comprises a bandage.

9. The wound dressing of claim 8, wherein the bandage is an acetate bandage.

10. The wound dressing of claim 7, wherein the honey composition is approximately 4 grams.
